(19) United States
(12) Patent Application Publication
(10) Pub. No.: US 2025/0134687 A1
(43) Pub. Date: May 1, 2025

(54) METHODS AND SYSTEMS FOR LIMB MOVEMENT DETECTION

(71) Applicant: Northwestern University, Evanston, IL (US)

(72) Inventors: Jun Yao, Naperville, IL (US); Julius Dewald, Downers Grove, IL (US); Jane Sullivan, Arlington Heights, IL (US)

(21) Appl. No.: 18/683,135

(22) PCT Filed: Aug. 9, 2022
(86) PCT No.: PCT/US2022/074678

§ 371 Date: Feb. 12, 2024

Related U.S. Application Data
(60) Provisional application No. 63/234,383, filed on Aug. 18, 2021.

(51) Int. Cl. A61F 5/01; A61B 5/00; A61B 5/395; A61B 5/397

(52) U.S. Cl. CPC A61F 5/0102; A61B 5/395; A61B 5/397; A61B 5/725

(57) ABSTRACT

A limb movement detection and stimulation system includes an orthosis configured to receive a limb of a patient. The system also includes one or more recording electrodes mounted to the orthosis and one or more stimulation electrodes mounted to the orthosis. The one or more stimulation electrodes are configured to stimulate the limb in response to the detected intended limb movement. The system also includes a processor operatively coupled to the orthosis. The processor is configured to determine. based on data sensed by the one or more recording electrodes. that the patient intends a limb movement. The processor is also configured to determine a movement state corresponding to the limb movement. and to energize the one or more stimulation electrodes to facilitate the movement state.

### CROSS-REFERENCE TO RELATED APPLICATION

The present application claims the priority benefit of U.S. Provisional Patent App. No. 63/234,383 filed on Aug. 18, 2021, the entire disclosure of which is incorporated herein by reference.

### REFERENCE TO GOVERNMENT RIGHTS

This invention was made with government support under grant number HD094073 awarded by the National Institutes of Health. The government has certain rights in the invention.

### BACKGROUND

It is estimated that approximately seventy-five percent of post-stroke survivors exhibit poor or no voluntary control of their paretic hand (i.e., the hand that was directly affected by the stroke). Currently, the evidence that conventional therapy produces meaningful hand function for more severely affected individuals is largely absent. Despite residual proximal arm movements, individuals without hand function typically do not incorporate the paretic arm in functional activities. This situation can lead to further disuse deficits, which can drastically affect quality of life.

### SUMMARY

An illustrative limb movement detection and stimulation system includes an orthosis configured to receive a limb of a patient, one or more recording electrodes mounted to the orthosis, one or more stimulation electrodes mounted to the orthosis, and a processor operatively coupled to the orthosis. The processor is configured to determine, based on data sensed by the one or more recording electrodes, that the patient intends a limb movement. The processor is also configured to determine a movement state corresponding to the limb movement. The processor is further configured to energize the one or more stimulation electrodes to facilitate the movement state.

The system can also include an accelerometer configured to detect a change in the relative positions of the one or more recording electrodes and the one or more stimulation electrodes with respect to a muscle that controls the limb. In an illustrative embodiment, upon being energized, the one or more stimulation electrodes stimulate the limb in response to the detected intended limb movement. In another embodiment, the data sensed by the one or more recording electrodes comprises raw electromyographic data. The system can also include a bandpass filter, and the processor can use the bandpass filter to filter the raw electromyographic data. The system can also include a coherence-based notch filter, and the processor can apply the coherence-based notch filter to the electromyographic data responsive to the determination that the patient intends the limb movement. In an illustrative embodiment, the coherence-based notch filter is user specific.

In another illustrative embodiment, the processor is configured to extract one or more features from the raw electromyographic data. The one or more extracted features can include one or more of an amplitude and a frequency. In another embodiment, the processor determines the movement state based at least in part on the one or more extracted features. The processor can compare the one or more extracted features to one or more thresholds to determines the movement state. The system can also include a transceiver operatively coupled to the processor, where the transceiver is configured to communicate with a remote computing device.

An illustrative method for performing limb movement detection and stimulation includes sensing data by one or more recording electrodes mounted to an orthosis that is configured to receive a limb of a patient. The method also includes determining, by a processor operatively coupled to the orthosis and based on the data sensed by the one or more recording electrodes, that the patient intends a limb movement. The method also includes determining, by the processor, a movement state corresponding to the limb movement. The method further includes energizing, by the processor, one or more stimulation electrodes mounted to the orthosis to facilitate the movement state.

The method can also include controlling, by the processor, an accelerometer mounted to the orthosis, wherein the processor controls the accelerometer to detect to detect a change in the relative positions of the one or more recording electrodes and the one or more stimulation electrodes with respect to a muscle that controls the limb. The method can also include stimulating, by the one or more stimulation electrodes, the limb in response to the detected intended limb movement. The method can also include applying a bandpass filter to the data sensed by the one or more recording electrodes. The method can also include applying a coherence-based notch filter to the data sensed by the one or more recording electrodes. The method can also include extracting, by the processor, one or more features from the data sensed by the one or more recording electrodes, where the one or more extracted features include one or more of an amplitude and a frequency. The method can further include determining, by the processor, the movement state based at least in part on the one or more extracted features.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIG. 1 depicts an example motor status chain for a daily reaching-grasping-retrieving task in accordance with an illustrative embodiment.

FIG. 2 depicts the algorithm for each of the transitions of FIG. 1 in accordance with an illustrative embodiment.

FIG. 3 is a table that compares the proposed ReIn hand device to other types of devices in accordance with an illustrative embodiment.

FIG. 4 is a table that describes components of the ReIn hand device in accordance with an illustrative embodiment.

FIG. 5 depicts typical EMG patterns of the tests performed on two impaired subjects in accordance with an illustrative embodiment.

FIG. 6 depicts results of the decision scheme for determining hand movements in accordance with an illustrative embodiment.

FIG. 7 is a block diagram of components included in a proposed ReIn-Hand arm-hand-orthosis system in accordance with an illustrative embodiment.

### DETAILED DESCRIPTION

Traditional research has suggested that, in individuals with mild stroke, successful hand function is best achieved through an intervention that is both intense and functional. However, current evidence for an effective intervention to regain hand function in individuals with moderate to severe post stroke hand paresis is almost absent. A possible contributor to such poor hand recovery in this population may be the inability to intensively practice with the paretic hand during activities of daily living. Such inability is largely due to paralysis and the expression of post-stroke abnormal muscle synergies. Specifically, abnormal synergies usually result in involuntary activity from wrist/finger flexors, making voluntary hand opening while reaching or lifting even more difficult than resting the paretic arm on a tabletop.

Using rehabilitation devices is a possible solution to enable individuals with moderate to severe stroke to use their paretic hand more intensely and in a functional context like reaching and grasping. Although many currently available hand rehabilitation devices support intense practice via repeated muscle activation, none of them sufficiently address the issue of abnormal muscle synergy, and thus cannot support intuitive hand control during functional arm movements like reaching. The inventors have thus proposed the development of a portable, synergy resistant, user-friendly, individualized, electromyographic (EMG)-driven functional electrical stimulation (FES) device that for the first time allows for reliable and intuitive control of the hand (i.e., ReIn-Hand) while using the paretic arm during lifting and reaching. Furthermore, to enable sufficient practice intensity, it is proposed to develop the rehabilitation device with home-use utilities, such that it can be used both in the clinic and at home. To implement this, described herein is a subject-specific arm/hand orthosis with embedded EMG recording electrodes and EMG stimulation electrodes.

As noted, multiple studies have suggested that the intensity and functional context in practice appear to be critical elements of successful interventions to improve hand function. Correspondingly, from a stroke survivors' perspective, the single most important factor impacting upper limb recovery is his/her use of the arm in everyday tasks. Unfortunately, a large number of stroke survivors lack sufficient hand control to engage in everyday task-related practice. This creates a clear gap between the need to promote functional arm/hand use and the capacity of those with severe arm paresis. Described herein is a device/algorithm to detect the users' intent of using the paretic hand and then use an artificial method, such as electric stimulation(ES) or a robot, to assist the post-stroke users to implement the desired tasks. Both electrical stimulation and robotic devices for driving a hand/arm are available in today's market. However, due to the lack of a detecting device/algorithm that can reliably detect a post-stroke user's intention during a functional task, a device that can help post-stroke users use their paretic arm/hand during every day operational tasks is still not available.

Applying EMG-based movement detection to individuals with stroke damage remains challenging. When detecting a single joint movement of the shoulder, elbow; wrist, or hand, recent work using surface electrodes achieved an accuracy of 46% in the paretic arm from 41 severe individuals, and of 86-90% using array electrodes (89 electrodes) in 20 mildly-severely impaired stroke survivors. However, EMG-based algorithms regarding hand movements while reaching or lifting in the stroke population have not been well investigated yet. The inventors have thus developed the only known algorithm capable of achieving an accuracy higher than 90% in the stroke population.

One key to success of the proposed system is that the inventors have reduced the complexity of the detection problem. It is well known that the complexity of a detection problem increases with the detection's dimension. For example, detecting 5 different motions based on activities from 8 muscles will be more complicated than detecting 2 motions. From this point of view, the simplest detection question has a binary output, i.e., a yes or no question. Therefore, one can break down a functional daily task into a transit between a series of motor statuses. In this way, the algorithms detect whether one should change to the following motor status at a specific time. That means a binary output at each time will tell the system whether to stay at the current status (e.g., detection output=‘No’) or change to the following status (e.g., detection output=‘Yes’).

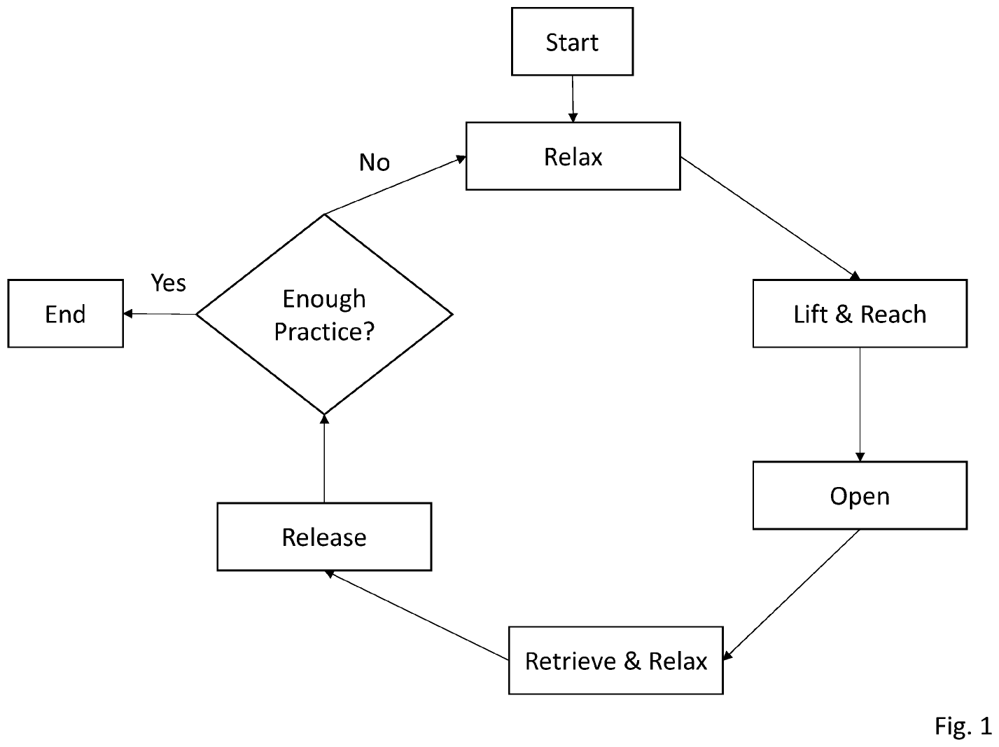

FIG. 1 depicts an example motor status chain for a daily reaching-grasping-retrieving task in accordance with an illustrative embodiment. In alternative embodiments, different elements may be included in the motor status chain and/or different types of tasks may be modeled. This design makes each detection the simplest version, although losing the flexibility of motor tasks. As shown in FIG. 1, since there is a fixed sequence, in each status, the real-time complex classification question is reduced to a 2-dimensional question of whether the subject intends to move the next status. A ‘yes’ response to the 2-dimensional question indicates that the algorithm should change to the next stage, and a ‘no’ response to the 2-dimensional question indicates that the algorithm should stay in the current stage. In FIG. 1, the transit (or transitions) are indicated by arrows, and the stages include start, relax, lift & reach, open, retrieve & relax, and release. A determination of whether enough practice has been completed is also made, and the algorithm repeats if additional practice is to be performed or ends if the practice is complete.

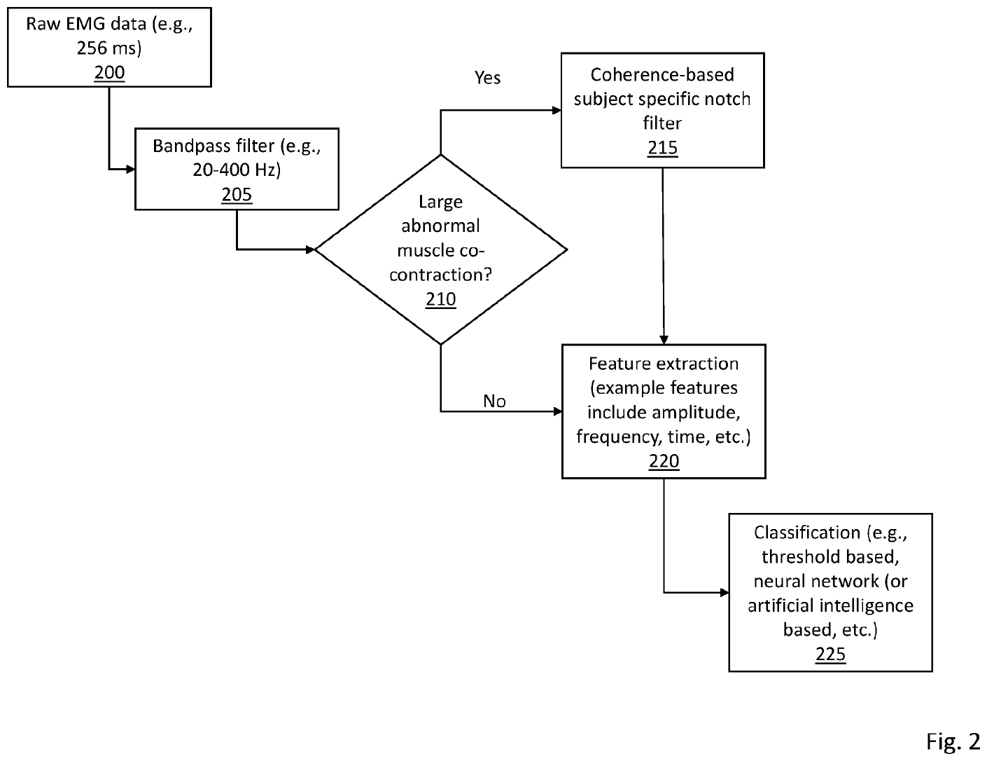

FIG. 2 depicts the algorithm for each of the transitions of FIG. 1 in accordance with an illustrative embodiment. In alternative embodiments, fewer, additional, and/or different operations may be included in the algorithm. In an operation 200, raw electromyographic (EMG) data is received from one or more sensors (i.e., recording electrodes) attached to the user. The raw EMG data can be received at an interval (or for a duration) of 256 milliseconds (ms) in one embodiment. In an operation 205, a bandpass filter is applied to the raw EMG data to filter out any interference, noise, etc. In one embodiment, the bandpass filter can be a 20-400 Hertz (Hz) bandpass filter, although in other embodiments a different frequency range may be used for the bandpass filter.

In an operation 210, a determination is made regarding whether a large abnormal muscle co-contraction occurred. Muscle co-contraction can be defined as the simultaneous muscle activation between agonist and antagonist muscles or between muscles across multiple joints. Following stroke, abnormal muscle co-contraction is commonly reported. For example, when driving shoulder abductors to lift the paretic arm, multiple flexors at the elbow; wrist, and finger will become active and thus generate involuntary flexion force. Clinically this is called flexion synergy, which means a stroke survivor cannot reach or open his/her hand when lifting against gravity. Co-contracted muscles are generated from a common neuron resource, and therefore a large coherence between co-contracted muscles can be detected at certain subject-specific frequencies. It is believed that voluntary muscle activities are contaminated by this involuntary co-contraction. If it is determined that a large muscle co-contraction did occur, the algorithm applies a coherence-based notch filter at the detected frequencies that are subject (or user) specific to the raw (bandpass filtered) EMG data in an operation 215 to remove the involuntary part of the muscle activities. If it is determined in the operation 210 that a large muscle co-contraction did not occur, the algorithm proceeds to an operation 220. The algorithm also proceeds to the operation 220 subsequent to applying the coherence-based subject specific notch filter in the operation 215. In the operation 220, feature extraction is performed to determine amplitude, frequency, timing, etc. of the filtered EMG data. In an operation 225, classification of the data is performed based on the feature extraction. The classification can be threshold-based (usually set as the upper or lower confidence intervals) or artificial intelligence (AI)-based using a neural network, depending on the implementation. In an illustrative embodiment, any of the operations of FIG. 2 can be performed by a device attached to the user and/or by a remote computing device that is in communication with the device attached to the user.

As discussed above, by fixing the sequence of a targeted functional daily task, one can simplify the detection problem with the cost of losing the flexibility of a random task. However, it has been determined that the gain is much more important than the loss for rehabilitation treatments, such as the post-stroke upper extremity function recovery. Reach-to-grasp movements are essential for everyday functions, such as retrieving objects, e.g., clothes, food, and drink, etc., and are used more frequently than other upper limb movements, such as gesturing, stabilizing objects, or for postural support. Therefore, upper limb task-specific training explicitly focusing on reach-to-grasp movements appears particularly relevant, as stroke survivors consulted about their goals for upper limb therapy programs prioritized activities involving reach-to-grasp.

Current hand function assistant devices can be generally divided into two categories: robotic device and functional electrical stimulation (FES) device. Robotic devices could possibly provide fine control of individual fingers. However, they are usually too large and too complex, and therefore are not suited for usage at home. In an illustrative embodiment, the proposed system can be implemented in the form of an FES device. Electrical stimulation devices can be subdivided into 4 categories, (1) simple surface electrical stimulation, (2) surface electrical stimulation coupled with a wrist hand orthosis, (3) electromyographic (EMG) triggered surface electrical stimulation, and (4) invasive electrical stimulation.

Simple surface electrical stimulation was first used to enhance arm function in the hand following stroke in the early 1960's. Numerous studies describe positive effects on decreasing impairment and enhancing arm function post stroke. The challenges to widespread use of surface stimulation systems include electrode placement, cumbersome equipment, the complexity of achieving consistent distal control, and lack of intuitive control. In addition, many systems are not easy for stroke survivors to apply without assistance, limiting use outside of the clinic. Surface electrical stimulation coupled with a wrist hand orthosis is another FES option. The biggest advantages of combining an electrical stimulation with a wrist hand orthosis (WFO) is that the system is portable and easy for users to manage.

Other FES devices have been developed to allow for triggering electrical stimulation using the user's EMG signal (EMG-triggered ES). When using these devices, the patient is asked to voluntarily contract the paretic muscles. When EMG activity exceeds a preset level, external electrical stimulation of the muscle takes place, which increases or triggers muscle contractions. The underlying thought behind intention-dependent, EMG triggered functional electrical stimulation is a positive influence on neuronal plasticity due to proprioceptive and somatosensory feedback from the electrically stimulated, active muscle contraction. Meta-analyses of clinical studies have demonstrated that triggered functional electrical stimulation appears to be superior to non-triggered electrical stimulation on motor control of the upper extremities, but cannot result in a clinically significant change in hand function. Part of the reason is that these commercially available EMG-triggered ES devices all allow for pre-set open or close without using the paretic arm. Furthermore, portability, ease of use, and cost are all barriers of these type devices to widespread use.

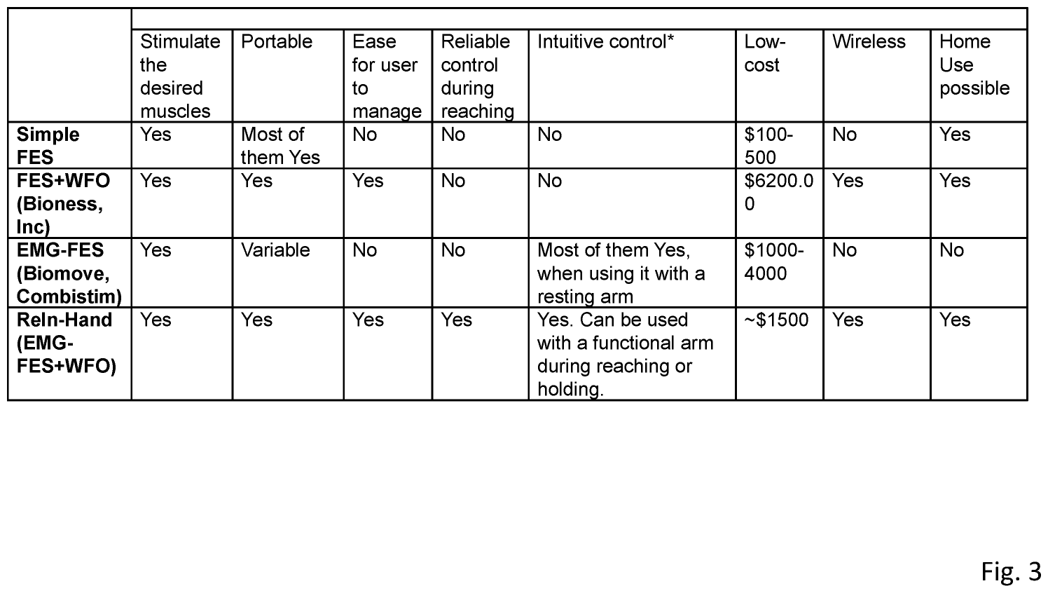

FIG. 3 is a table that compares the proposed hand rehabilitation (ReIn) device to other types of devices in accordance with an illustrative embodiment. In the table of FIG. 3, intuitive control can be defined as the stimulation to the desired muscles that will be turned on based on subject's intention of using these muscles. For example, finger extensors will be turned on when subject wants to open his/her hand. Furthermore, this type of control involves limited time to learn (e.g., about 2 hours). As shown, the proposed ReIn device is able to stimulate the desired muscles, is portable, is easy to a user to manage, provides reliable control during a reaching movement, has intuitive control, is low cost, has wireless capability, and is possible to use in home. None of the existing systems have all of this functionality.

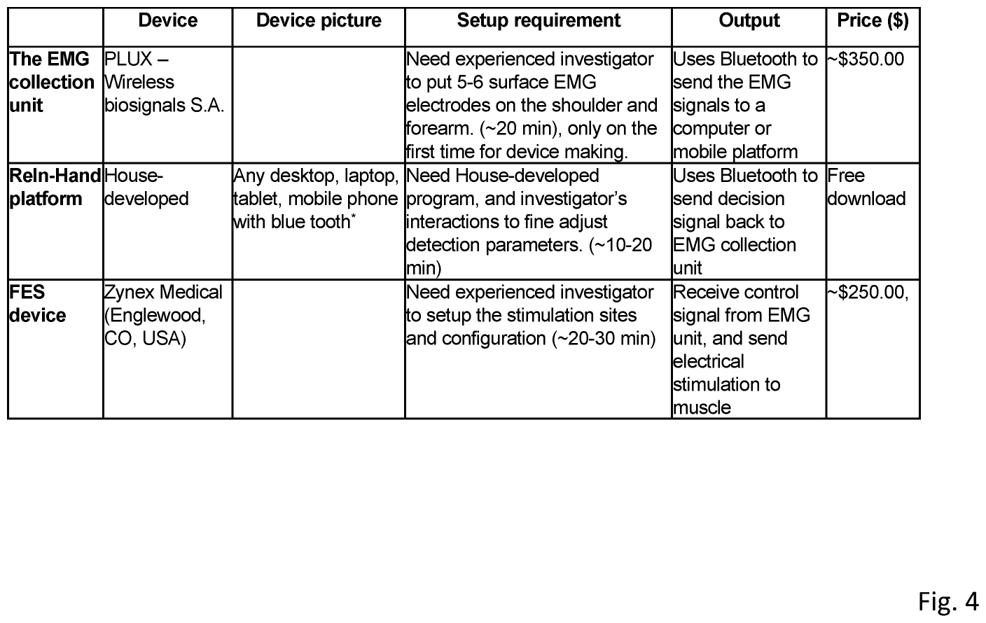

In an illustrative embodiment, the proposed ReIn-Hand rehabilitation device includes an EMG data collection unit, an EMG data processing core (ReIn-Hand real time platform), and an output unit (FES device). FIG. 4 is a table that describes components of the ReIn hand device in accordance with an illustrative embodiment. In alternative embodiments, fewer, additional, and/or different device components may be used. With the proposed EMG unit, the system can be run on devices that use Bluetooth supporting serial peripheral interface (SPI) mode, like any devices using an Android operating system. Alternatively, a different EMG unit may be used and/or different and additional devices may be used to implement the system. As described in more detail below, a prototype device was made by the inventors, and the prototype device included a house-developed ReIn-Hand real time EMG platform.

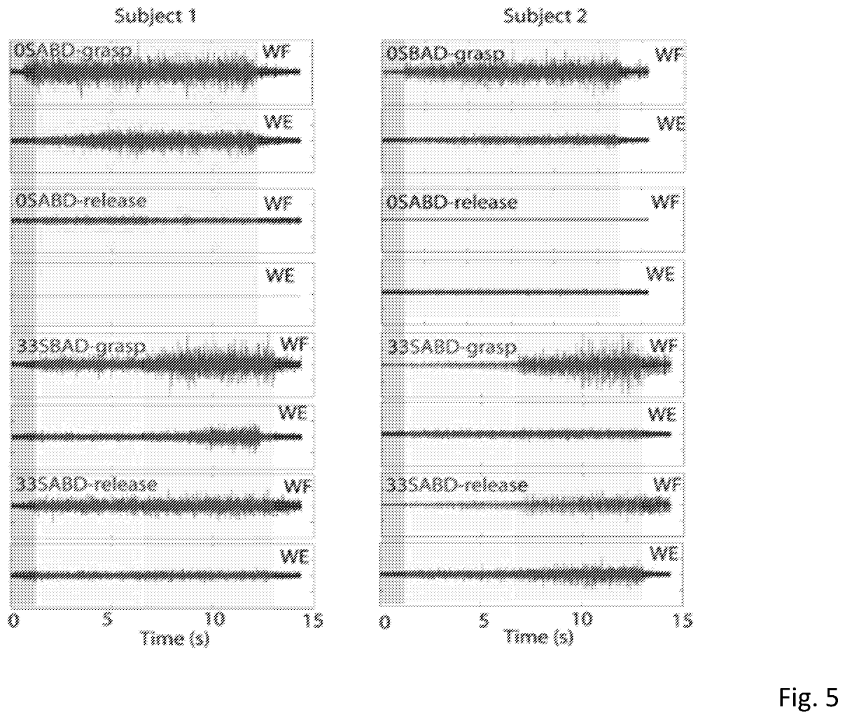

Applying EMG-based movement detection to individuals with stroke remains challenging. Typical EMG patterns when performing maximal level grasp and release with the paretic arm generating 33% of their maximal voluntary shoulder abduction (33SABD-grasp) or resting on a table (‘0SABD-grasp’) was tested in 2 severely impaired individuals using the prototype device. FIG. 5 depicts typical EMG patterns of the tests performed on two impaired subjects in accordance with an illustrative embodiment. As shown in FIG. 5, during a first (shaded) time window; subjects were relaxing on a table preparing for the requested movements. During the last (white) time window, subjects relaxed on the table again.

Referring to FIG. 5, due to muscle weakness, no clear EMG activity was observed from the wrist extensors (WE) in any movement for subject 2 even though this subject was performing maximal hand open to release. The challenge induced by the increased level of co-activation between antagonist and agonist can be seen in the ‘0SABD-grasp’ case of subject 1, where similar level of activities were recorded from both WE and wrist flexor (WF), making the EMG patterns between different movements less distinguishable. Furthermore, the abnormal muscle synergy across multiple joints can be seen in the ‘33SABD-release’ case for subject 1. Specifically, clear activity from WF can be observed during the lifting up time window, and no more activity from WE can be seen during the hand-opening phase. All of these results demonstrate that using EMG amplitudes to detect hand movements in more severely impaired subjects is difficult due to abnormal synergies and muscle weakness.

The inventors have therefore successfully developed novel algorithms to detect hand grasp and release during lifting the paretic arm and/or reaching. In an effort to detect voluntary hand movements, the inventors collected EMGs from 4-7 different muscles from test candidates: first dorsal interossei, flexor pollicis brevis, extensor digitorum communis, extensor carpi radialis, flexor digitorum profundus, flexor carpi radialis, lateral head of the triceps, bicep brachii, intermediate deltoid, and posterior deltoid. A 250 millisecond (ms) long window with increments of 50 ms of raw EMG signals was processed at one time to calculate four EMG features (mean, absolute value, zero crossing, slope, sign changes, and waveform length). A linear discriminant classifier, which is a linear combination of features that separate 2 or more classes of events, is used for classifications of movement intentions in each of the time windows.

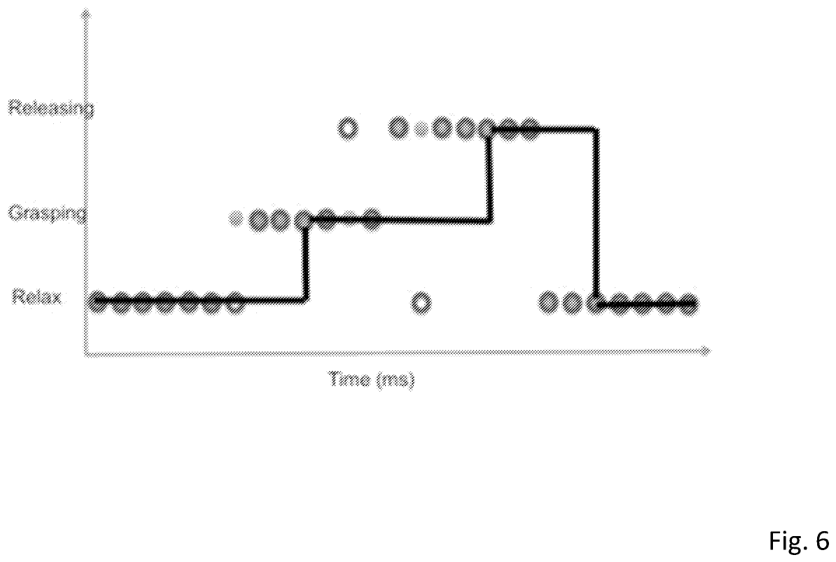

If three continuous windows make a consistent decision, a final decision for the hand movements is sent to the ES device (see FIG. 6). FIG. 6 depicts results of the decision scheme for determining hand movements in accordance with an illustrative embodiment. The dots and circles are real hand movements and detected hand movements, respectively, during each moving window. The black line shows the control that is sent to the ES device. This decision scheme will further increase the reliability/accuracy of the detection with the cost of a minimum delay at 350 ms, which is durable with more severely impaired individuals. This detection method has been adopted to predict movement intentions and proven to be efficient in pattern recognition. Using EMG signals from multiples muscles, including both proximal and distal muscles, for movement intent detection is novel. Due to the post-stroke synergy-induced muscle activity, approaches detecting changes in EMG-amplitude of a single muscle have little chance to be successful in hand-control when lifting up the arm against gravity or reaching towards an object.

The inventors have also demonstrated that shoulder abduction decreases the detection rate of hand opening in individuals with moderate to severe stroke, thus making the detection of hand opening signals during functional arm movements very challenging. Innovatively, the inventors have thus designed algorithms using signals from both proximal and distal muscles to deal with the impact of abnormal synergic muscle activation for the first time, thus guaranteeing the detection of a grasp to release during a functional arm movement. The proposed device is the only known device that can detect a hand opening during a functional arm movement.

In an illustrative embodiment, the proposed ReIn-Hand device can be mobile phone based with a user-friendly interface that allows the user to fine-adjust the detection parameters. Furthermore, a subject-specific orthosis with all of the EMG recording and stimulation electrodes embedded has been developed to reduce the difficulty in setup and save setup time. Additionally, the device can include an accelerometer to detect the change in the relative positions of surface recording and stimulation electrodes in regard to the desired muscle, thus allowing for the position-dependent auto-selection of the recording and stimulation electrodes.

In one embodiment, the proposed ReIn-Hand rehabilitation device can be a portable device with user-friendly interfaces (e.g., a smartphone, smart watch, tablet laptop computer, etc.). The device can include automatic parameter adjustment and friendly graphical user interfaces (GUIs). The user interfaces can be implemented using one or more displays, one or more touch screens, a mouse, a keyboard, buttons, etc. The GUI can include an interface for the clinician to ensure EMG recordings. The clinician can, for example, start a user with 5 EMG recording electrodes to detect the hand-opening intention with various functional arm movements. During this initial phase, the clinician can ask a user to perform a sequence of pre-set movements (reaching for a jar, grasping, retrieving, and opening it, or grasping a fork and stabilizing a piece of play-Doh, while using the non-affected hand to cut the play-Doh, etc.). Recorded EMGs will be shown as a time series with various colors representing the activity during different movements. This will allow the clinician to ensure the correct site of the recording electrodes and good contact between the electrodes and the skin, allowing for proper measurement of the EMG activity.

The GUI can also include an interface to adjust the classifier. The ReIn-Hand data processing core processes the recorded EMGs to extract several features from each of the EMG-channels for each of the movements. Features can be selected automatically to maximize the successful detection of hand movements. The processing core can also generate a set of rules for classification. Once the rules are available, a user can use the ReIn-Hand device to perform the pre-set movement. The GUI can provide real time feedback of the selected features, and the system can collect either touch-screen or voice feedback from the clinician or the user at the end of each trial for the purpose of fine-adjusting the rules. The voice-control will allow easy-use for patients with hand paresis. Feedback can include false-stimulation (i.e., detecting an opening when the user is not attempt to open his/her hand), cannot-trigger (i.e., cannot detect an opening when the user is attempting to open the hand), perfect performance, etc. Using the feedback, the system will automatically adjust the rules, or create new rules if needed, until the user can perform the movements very well. The preliminary results suggested that a set of rules usually could be used for 7-10 days with fine-adjustments, but without the need for a new set of rules.

The GUI can also include an interface for the user to set up the device. In one embodiment, the interface to guide the user to setup the ReIn-Hand device can include a sequence of setting up the orthosis and the stimulator (including setting up the stimulation amplitude and configuration of an e-Wave stimulator), connecting the EMG-recording unit (e.g., Muscle BIT) to EMG recording electrodes with a single combined cable, and connecting the stimulator (e-Wave stimulator) to the EMG stimulation electrodes.

The GUI can also include an interface for the user to use the device. Once set up, the user will perform the pre-selected movements. The user will provide myoelectric biofeedback using the similar GUI as mentioned herein. Upon receiving the feedback of ‘cannot-trigger’, the data processing core can automatically perform a fine-adjustment of the current rules. Upon receiving feedback of ‘setting new rules’, the data processing core can stop and notify the user to quit/shut down the whole device, waiting for the clinician to re-check it.

The device can also include an interface for safety control. The safety control can be available in every single GUI to allow the user or clinician to stop the whole system. Although the e-Wave Complete Electrotherapy System is an FDA approved device, which has very rare risk, a safety control can be used to prevent any potential harm. Such safety control also includes a module to guarantee no less than an 8:1 ratio of resting/stimulation time and no more than 1-hour usage during a single session to avoid muscle fatigue. Alternatively, different resting/stimulation and usage times may be utilized. The GUI and its features can be implemented in an application that can be run on a mobile device, such that the overall system is able to provide safe, reliable, and intuitive hand control during functional arm movement for individuals with moderate to severe stroke.

The proposed device has been implemented as a subject-specific arm-hand-orthosis with all EMG-recording and stimulation electrodes embedded. During one of the initial visits of the patient, the clinician will detect the EMG-recording/stimulation sites with various arm configurations. Once determined, these positions can be marked. Then, the whole forearm and hand (if needed) can be scanned (e.g., by a David-3D Sls-2 scanner (Koblenz and Brounschweig, Germany). A technician can then use the scan output to design a subject-specific arm-hand-orthosis (AHO), keeping all the positions for electrodes empty, and all positions for wires dented (with reduced thickness). The designed AHO can then be printed out (e.g., by an Ultimaker 2 Extended 3D printer) or otherwise manufactured.

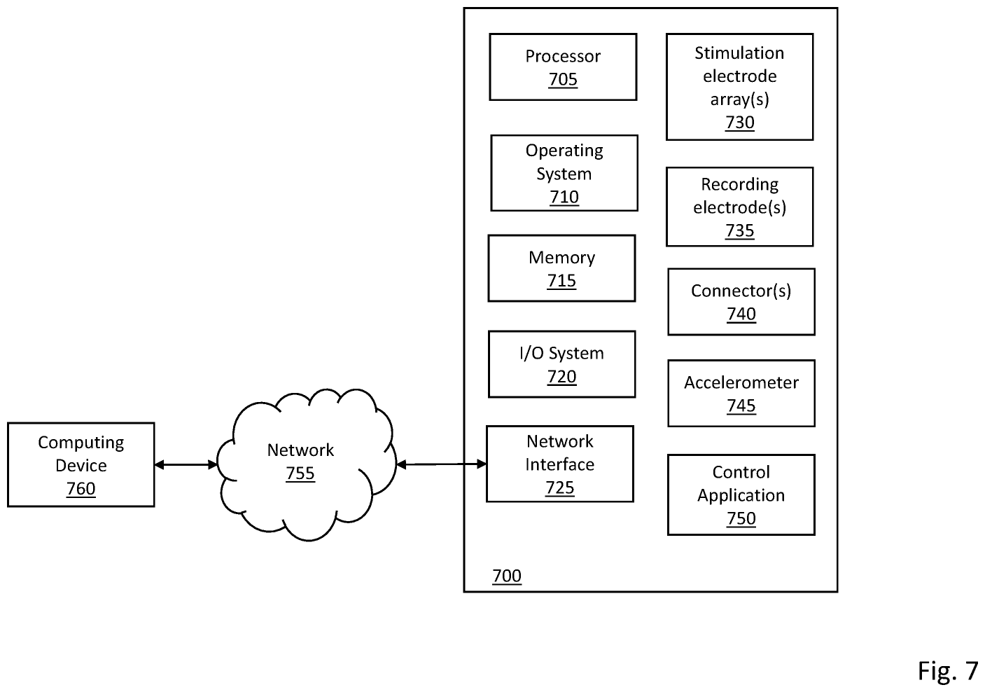

Subsequently, a technician can embed all the EMG-recording and stimulation electrodes into the AHO and combine various cables to two connectors: one for data recording and one for stimulation. FIG. 7 is a block diagram of components included in a proposed ReIn-Hand arm-hand-orthosis system in accordance with an illustrative embodiment. More specifically, FIG. 7 shows a hand rehabilitation orthosis device 700 in direct or indirect communication with a network 755. In implementation of the device of FIG. 7, all of the different electrodes are attached to a printed (or otherwise fabricated) AHO device.

The orthosis device 700 includes a processor 705, an operating system 710, a memory 715, an input/output (I/O) system 720, a network interface 725, stimulation electrode array(s) 730, recording electrode(s) 735, connector(s) 740, an accelerometer 745, and a control application 750. In alternative embodiments, the orthosis device 700 may include fewer, additional, and/or different components. The components of the orthosis device 700 communicate with one another via one or more buses or any other interconnect system. In an illustrative embodiment, the orthosis device 700 can be a printed device to which the stimulation electrode array(s) 730, the recording electrode(s) (735), and other components are mounted. Alternatively, any of the non-sensor components of the orthosis device 700 may be remotely positioned on a computing device 760 that is accessible through the network 755.

The processor 705 of the orthosis device 700 can be in electrical communication with and used to perform any of the operations described herein, such as gathering raw data, processing the gathered data, sending data to external systems, etc. The processor 705 can be any type of computer processor known in the art, and can include a plurality of processors and/or a plurality of processing cores. The processor 705 can include a controller, a microcontroller, an audio processor, a graphics processing unit, a hardware accelerator, a digital signal processor, etc. Additionally, the processor 705 may be implemented as a complex instruction set computer processor, a reduced instruction set computer processor, an x86 instruction set computer processor, etc. The processor 705 is used to run the operating system 710, which can be any type of operating system.

The operating system 710 is stored in the memory 715, which is also used to store programs, algorithms, network and communications data, peripheral component data, the control application 750, and other operating instructions. The memory 715 can be one or more memory systems that include various types of computer memory such as flash memory, random access memory (RAM), dynamic (RAM), static (RAM), a universal serial bus (USB) drive, an optical disk drive, a tape drive, an internal storage device, a non-volatile storage device, a hard disk drive (HDD), a volatile storage device, etc.

The I/O system 720, or user interface, is the framework which enables users (and peripheral devices) to interact with the orthosis device 700. In alternative embodiments, the I/O system 720 can be on a remote computing device, such as the computing device 760. The I/O system 720 can include one or more keys or a keyboard, one or more buttons, a speaker, a microphone, etc. The I/O system 720 allows the user to interact with and control the orthosis device 700. The I/O system 720 can also include circuitry and a bus structure to interface with and control peripheral computing components such as one or more power sources, etc.

The network interface 725 includes transceiver circuitry (e.g., a receiver and/or a transmitter) that allows the orthosis device 700 to transmit and receive data to/from other devices such as the computing device 760. The computing device 760 can be a user device such as a cell phone, tablet, laptop computer, etc. The computing device 760 can also be in the form of one or more remote computing systems, servers, websites, etc. The network interface 725 enables communication through the network 755, which can be one or more communication networks. The network 755 can include a cable network, a fiber network, a cellular network, a wi-fi network, a landline telephone network, a microwave network, a satellite network, etc. The network interface 725 also includes circuitry to allow device-to-device communication such as near field communication (NFC), Bluetooth® communication, etc. In alternative embodiments, the orthosis device 700 may be a standalone system that does not connect to the network 755.

The orthosis device 700 also includes one or more stimulation electrode arrays 730. As discussed herein, the one or more stimulation electrode arrays can be used to stimulate muscles in the hand/arm of the user to assist with motor function. In one embodiment, a first stimulation electrode array can be positioned over the back of the hand of the user, and one or more additional stimulation electrode arrays can be positioned over muscles in the user's hand and/or forearm. In one embodiment, a stimulation electrode array can be positioned over each of the following muscle groups: first dorsal interossei, flexor pollicis brevis, extensor digitorum communis, extensor carpi radialis, flexor digitorum profundus, flexor carpi radialis, lateral head of the triceps, bicep brachii, intermediate deltoid, and posterior deltoid.

The orthosis device 700 further includes one or more recording electrodes 735 and one or more connectors 740. The one or more recording electrodes 735 are used to record the raw data that is used to monitor and detect intended limb movement. The recording electrodes 735 can be positioned proximate to the stimulation electrode arrays 730 (e.g., on or near any of the muscles listed above) and/or anywhere else on the orthosis device 700. The one or more connectors 740 are used to connect the various electrodes and other components of the device. In one embodiment, the one or more connectors 740 can be implemented as junction boxes that receive wires from the various device components.

The orthosis device 700 also includes an accelerometer 745. Alternatively, a plurality of accelerometers may be included on the device. In an illustrative embodiment, the accelerometer is included to detect forearm rotation of the user based on relative positions of the various sensors mounted to the device. Based on the rotation information, different combinations of stimulation electrodes may be used. As one example, when more pronation is detected, multiple arrays of stimulation electrodes can be used. However, when more supination is detected, only a single array of stimulation electrode may be used.

The control application 750 can include software and algorithms (e.g., in the form of computer-readable instructions) which, upon activation or execution by the processor 705, performs any of the various operations described herein such as activating stimulation electrodes, using the recording electrodes to record raw data, filtering the raw data, determining whether a large muscle contraction has occurred, performing feature extraction from the data, classifying the data, communicating with remote computing devices, etc. In an alternative embodiment, the filters described herein may be implemented as hardware that is incorporated into the orthosis device 700 or the computing device 760. The control application 750 can utilize the processor 705 and/or the memory 715 as discussed above.

The inventors have tested the therapeutic value of the proposed orthosis device in a pilot study involving 8 individuals with moderate to severe stroke. Six subjects have finished a 7-week intervention. Among them, 5 of them exhibit increased hand function, and one of them has a clinically significant increase of hand function. Given that none of the previous studies/devices have shown to be effective in regaining hand function in individuals with moderate to severe stroke, these outcomes are very promising.

The proposed system can be used as a treatment device for individuals with moderate to severe stroke to participate intensive practice in a functional context either at clinical practice or at home. Functional practice has been recognized as critical for an effective intervention. Most of functional practice involves the use of the hand together with other functional arm activities, such as lifting the arm against the gravity or reaching towards an object. Although regaining function of the paretic arm seems to be promising, when considering using a paretic hand during functional arm activities, abnormal muscle synergies usually result in involuntary wrist/finger flexors activity. This makes voluntary hand-opening difficult and also challenges the effectiveness of a hand rehabilitation device. Currently, none of the traditionally available hand rehabilitation devices sufficiently address hand control in the context of synergies. Thus, the proposed device is critical in that it is the first device that permits for reliable and intuitive hand opening while using the paretic arm to lift or to reach during activities of daily living.

The proposed system can also be used as a neuroprosthesis for individuals with moderate to severe stroke for hand control during daily living at home. Due to shortened inpatient rehabilitation stays and reduced funding for outpatient rehabilitation, therapists have been forced to focus less on the use of the upper extremity. The limited resources and other practical reasons (e.g. difficulty in arranging transportation, limited time with physical therapist, etc.) make it more beneficial to this population, if such a rehabilitation device is subject-specific, easy-to-use, low-cost, and feasible to use at home. Treatment effects of any rehabilitation devices will be subject-specific, varying due to the lesion-size, lesion-site, and many other factors. In the case that a subject cannot regain the voluntary hand control, s/he can use this device as a neuroprosthesis during activities of daily living.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more”.

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A limb movement detection and stimulation system comprising:
an orthosis configured to receive a limb of a patient:
one or more recording electrodes mounted to the orthosis:
one or more stimulation electrodes mounted to the orthosis; and
a processor operatively coupled to the orthosis, wherein the processor is configured to:
determine, based on data sensed by the one or more recording electrodes, that the patient intends a limb movement:
determine a movement state corresponding to the limb movement; and
energize the one or more stimulation electrodes to facilitate the movement state.

2. The system of claim 1, further comprising an accelerometer configured to detect to detect a change in the relative positions of the one or more recording electrodes and the one or more stimulation electrodes with respect to a muscle that controls the limb.

3. The system of claim 1, wherein, upon being energized, the one or more stimulation electrodes stimulate the limb in response to the detected intended limb movement.

4. The system of claim 1, wherein the data sensed by the one or more recording electrodes comprises raw electromyographic data.

5. The system of claim 4, further comprising a bandpass filter, wherein the processor uses the bandpass filter to filter the raw electromyographic data.

6. The system of claim 5, further comprising a coherence-based notch filter, wherein the processor applies the coherence-based notch filter to the electromyographic data responsive to the determination that the patient intends the limb movement.

7. The system of claim 6, wherein the coherence-based notch filter is user specific.

8. The system of claim 4, wherein the processor is configured to extract one or more features from the raw electromyographic data.

9. The system of claim 8, wherein the one or more extracted features include one or more of an amplitude and a frequency.

10. The system of claim 8, wherein the processor determines the movement state based at least in part on the one or more extracted features.

11. The system of claim 10, wherein the processor compares the one or more extracted features to one or more thresholds to determines the movement state.

12. The system of claim 1, further comprising a transceiver operatively coupled to the processor, wherein the transceiver is configured to communicate with a remote computing device.

13. A method for performing limb movement detection and stimulation, the method comprising:
sensing data by one or more recording electrodes mounted to an orthosis that is configured to receive a limb of a patient;
determining, by a processor operatively coupled to the orthosis and based on the data sensed by the one or more recording electrodes, that the patient intends a limb movement;
determining, by the processor, a movement state corresponding to the limb movement; and
energizing, by the processor, one or more stimulation electrodes mounted to the orthosis to facilitate the movement state.

14. The method of claim 13, further comprising controlling, by the processor, an accelerometer mounted to the orthosis.

15. The method of claim 14, wherein the processor controls the accelerometer to detect to detect a change in the relative positions of the one or more recording electrodes and the one or more stimulation electrodes with respect to a muscle that controls the limb.

16. The method of claim 13, further comprising stimulating, by the one or more stimulation electrodes, the limb in response to the detected intended limb movement.

17. The method of claim 13, further comprising applying a bandpass filter to the data sensed by the one or more recording electrodes.

18. The method of claim 13, further comprising applying a coherence-based notch filter to the data sensed by the one or more recording electrodes.

19. The method of claim 13, further comprising extracting, by the processor, one or more features from the data sensed by the one or more recording electrodes, wherein the one or more extracted features include one or more of an amplitude and a frequency.

20. The method of claim 19, further comprising determining, by the processor, the movement state based at least in part on the one or more extracted features.
